(19) United States
(12) Patent Application Publication
(10) Pub. No.: US 2026/0062373 A1
(43) Pub. Date: Mar. 5, 2026

(54) MIXED ESTERS OF CYCLOHEXANE-1,2,4-TRIPROPIONIC ACID AND A PLASTICIZER COMPOSITION COMPRISING SAID MIXED ESTERS

(71) Applicant: Evonik Oxeno GmbH & Co. KG, Marl (DE)

(72) Inventors: Michael Grass, Haltern am See (DE); Monika Rusek, Essen (DE); Michael van Eickels, Recklinghausen (DE)

(73) Assignee: Evonik Oxeno GmbH & Co. KG, Marl (DE)

(21) Appl. No.: 19/316,059

(22) Filed: Sep. 2, 2025

(30) Foreign Application Priority Data
Sep. 3, 2024 (EP) 24198041.6

(51) Int. Cl. C07C 69/013; C08K 5/12

(52) U.S. Cl. CPC C07C 69/013; C08K 5/12

(57) ABSTRACT

A plasticizer composition contains a compound of the formula (1)

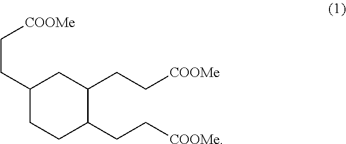 [SMILES: COC(=O)CCC1CCC(CCC(=O)OC)C(CCC(=O)OC)C1]

and at least one dialkyl terephthalate, where the two alkyl groups in the dialkyl terephthalate each have 8 or 9 carbon atoms.

### CROSS-REFERENCE TO RELATED APPLICATIONS

This patent application claims priority to European Patent Application No. 24198041.6, filed on Sep. 3, 2024, in the European Patent Office, the entire disclosure of which is hereby incorporated by reference herein.

### BACKGROUND OF THE INVENTION

### Field of the Invention

The invention provides a mixture of triesters of cyclohexane-1,2,4-tripropionic acid of the following formula (1):

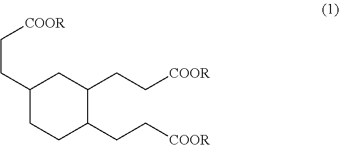

wherein 5 to 75 mol % of the R radicals are methyl radicals and 25 to 95 mol % of the R radicals are a C2- to C10-alkyl radical.

### Description of Related Art

The inventive compounds of the formula (1) are mixed esters of cyclohexane-1,2,4-tripropionic acid. Trimethyl esters of cyclohexane-1,2,4-tripropionic acid in which all R radicals are methyl radicals are known in principle and have been described, for example, in EP 3 838 886 A1. It was also mentioned therein that these trimethyl esters of cyclohexane-1,2,4-tripropionic acid can be used as plasticizers. The trimethyl ester has been described in this context as being a product having low gelation temperature.

The trimethyl esters of cyclohexane-1,2,4-tripropionic acid do not always show solely positive properties when used as plasticizers in plastics. If the trimethyl ester were to be used as plasticizer, for example in a PVC-based plastisol formulation, the plastisol would have quite a high viscosity. In addition, when the plastisol is processed, a not inconsiderable proportion of the trimethyl ester would evaporate and hence no longer remain in the end product.

### SUMMARY OF THE INVENTION

It was thus an object of the present invention to provide a mixed ester having better performance properties than the trimethyl ester of cyclohexane-1,2,4-tripropionic acid.

The invention includes but is not limited to the following embodiments:
1. A mixture of triesters of cyclohexane-1,2,4-tripropionic acid of the following formula (1):

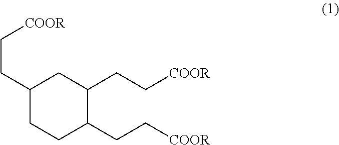

wherein 5 to 75 mol % of the R radicals in the mixture are methyl radicals and 25 to 95 mol % of the R radicals are a C2- to C10-alkyl radical.
2. The mixture according to Embodiment 1, wherein 15 to 70 mol % of the R radicals in the mixture are methyl radicals and 30 to 85 mol % of the R radicals are a C2- to C10-alkyl radical.
3. The mixture according to Embodiment 1, wherein the C2- to C10-alkyl radical is a C5- to C9-alkyl radical.
4. A plasticizer composition, comprising a mixture of triesters of cyclohexane-1,2,4-tripropionic acid of the following formula (1):

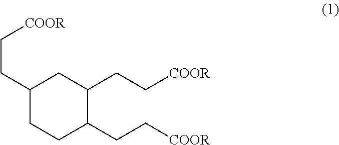

wherein
5 to 75 mol % of the R radicals in the mixture are methyl radicals and 25 to 95 mol % of the R radicals are a C2- to C10-alkyl radical, and
at least one further plasticizer.
5. The plasticizer composition according to Embodiment 4, wherein the mixture of triesters of the cyclohexane-1,2,4-tripropionic acid of the formula (1) and the further plasticizer in the plasticizer composition are in a weight ratio (mixture of triesters of the cyclohexane-1,2,4-tripropionic acid of the formula (1): further plasticizer) of 1:99 to 99:1.
6. The plasticizer composition according to Embodiment 4, wherein the at least one further plasticizer is selected from the group consisting of adipates, benzoates, chlorinated hydrocarbons, citrates, epoxidized fatty acid esters, epoxidized vegetable oils, epoxidized acylated glycerides, furandicarboxylates, phosphates, succinates, sulfonamides, sulfonates, terephthalates, isophthalates, cyclohexanedicarboxylates, trimellitates, phthalates, and oligomeric or polymeric esters based on adipic acid, succinic acid or sebacic acid.
7. The plasticizer composition according to Embodiment 4, wherein the plasticizer composition additionally comprises an epoxidized oil or an epoxidized alkyl ester of a fatty acid.
8. The plasticizer composition according to Embodiment 7, wherein the epoxidized oil is selected from the group consisting of epoxidized soybean oil, epoxidized castor oil, epoxidized linseed oil, epoxidized palm oil, epoxidized stearate, epoxidized oleate, epoxidized tall oil, epoxidized linoleate and mixtures thereof.
9. The plasticizer composition according to Embodiment 8, wherein the epoxidized oil is epoxidized soybean oil or epoxidized linseed oil.
10. The plasticizer composition according to Embodiment 7, wherein the epoxidized oil is present in the plasticizer composition in an amount of 1 to 150 parts by weight based on 100 parts by weight of a sum total of a compound of formula (1) and a dialkyl terephthalate.
11. A plastics composition, comprising:
the mixture according to Embodiment 1, and
a plastic.
12. The plastics composition according to Embodiment 11, wherein a proportion of the mixture is 5 to 150 parts by weight per 100 parts by weight of plastic.
13. The plastics composition according to Embodiment 11, wherein the plastic is selected from the group consisting of polyvinylchloride (PVC), polyalkylmethacrylate (PAMA), polyvinylbutyral (PVB), polyurethane, polysulfides, polylactic acid (PLA), polyhydroxybutyral (PHB), nitrocellulose and copolymers of vinyl chloride with vinyl acetate or with butyl acrylate.
14. The plastics composition according to Embodiment 13, wherein the plastic is polyvinylchloride (PVC).
15. A product, comprising:
the plastics composition according to Embodiment 11,
wherein the product is selected from the group consisting of adhesives, sealing compounds, coating compounds, varnishes, paints, plastisols, foams, imitation leathers, floor coverings, roofing membranes, underbody protection, fabric coatings, cables, wire insulation, hoses, extruded articles, films, automotive interior components, wallpaper, inks, toys, contact sheets, food packaging, and medical articles.

### DETAILED DESCRIPTION OF THE INVENTION

This object is achieved by the mixed ester according to Embodiment 1. The invention accordingly provides a mixture of triesters of cyclohexane-1,2,4-tripropionic acid of the following formula (1):

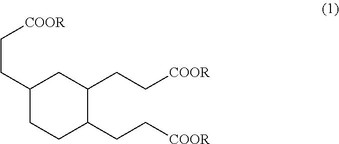

wherein 5 to 75 mol % of the R radicals in the mixture are methyl radicals and 25 to 95 mol % of the R radicals are a C2- to C10-alkyl radical. Preferred embodiments of the present invention are specified in the subsidiary embodiments. The reported percentages (mol %) each refer to all R radicals in the mixture.

A preferred embodiment of the present invention relates to a mixture of triesters of cyclohexane-1,2,4-tripropionic acid according to the following formula (1):

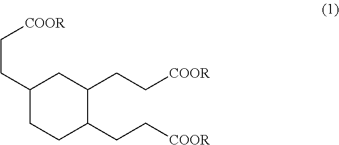

wherein 15 to 70 mol %, more preferably 20 to 65 mol %, of the R radicals in the mixture are methyl radicals and 30 to 85 mol %, more preferably 35 to 80 mol %, of the R radicals are C2- to C10-alkyl radicals. The percentage in mol % relates here too in each case to 100 mol % of the R radicals, i.e. all R radicals in the compound of formula (1) in the mixture.

The C2- to C10-alkyl radical present in the described proportion in the triesters of cyclohexane-1,2,4-tripropionic acid of formula (1) in the mixture according to the invention consists of ethyl groups, propyl groups, butyl groups, pentyl groups, hexyl groups, heptyl groups, octyl groups, nonyl groups or decyl groups. The alkyl groups mentioned include all isomers of the corresponding alkyl group and mixtures of different isomers of the corresponding alkyl group.

The term “pentyl groups” includes, for example, at least the 2-methylbutyl group, the n-pentyl group and the 3-methylbutyl group. “Nonyl groups” include, for example, n-nonyl groups or isononyl groups. For the purposes of the present invention, an isononyl group means a mixture of linear and branched C9-alkyl groups.

In a preferred embodiment, the C2- to C10-alkyl radical is a C4- to C9-alkyl radical, more preferably a C5- to C9-alkyl radical. A preferred embodiment of the present invention thus relates to a mixture of triesters of cyclohexane-1,2,4-tripropionic acid according to the following formula (1):

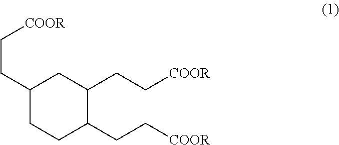

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C5- to C9-alkyl radical, more preferably a C4- to C9-alkyl radical. The percentage (mol %) also refers here in each case to all R radicals in the mixture.

The C4- to C9-alkyl radical or C5- to C9-alkyl radical present in the described proportion in the triesters of cyclohexane-1,2,4-tripropionic acid of formula (1) in the preferred mixture according to the invention consists of butyl groups, pentyl groups, hexyl groups, heptyl groups, octyl groups, nonyl groups. The alkyl groups mentioned include all isomers of the corresponding alkyl group and a mixture of different isomers of the corresponding alkyl group.

In a particularly preferred embodiment of the present invention, the C2- to C10-alkyl radical is a pentyl group or an isononyl group. The term “pentyl groups” includes, for example, at least the 2-methylbutyl group, the n-pentyl group and the 3-methylbutyl group or mixtures thereof. For the purposes of the present invention, an isononyl group means a mixture of linear and branched C9-alkyl groups.

A particularly preferred embodiment of the present invention thus relates to a mixture of triesters of cyclohexane-1,2,4-tripropionic acid according to the following formula (1):

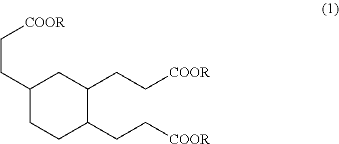

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a pentyl group or an isononyl group. The percentage (mol %) also refers here to all R radicals in the mixture.

The mixture of triesters of cyclohexane-1,2,4-tripropionic acid is currently not commercially available. But preparation is possible via transesterification of the trimethyl ester of cyclohexane-1,2,4-tripropionic acid with an appropriate substoichiometric amount of a C2 to C10 alcohol, preferably a C4 to C9 alcohol, more preferably a C5 to C9 alcohol and most preferably pentanol or isononanol. A process for preparing the trimethyl esters of cyclohexane-1,2,4-tripropionic acid is described, for example, in EP 3 842 411 A1. The esters of cyclohexane-1,2,4-tripropionic acid are thus generally obtainable.

The present invention also provides a plasticizer composition comprising a mixture of triesters of cyclohexane-1,2,4-tripropionic acid of the following formula (1):

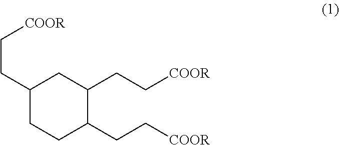

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group, and at least one further plasticizer. The abovementioned percentages (mol %) here too relate in each case to all R radicals in the mixture.

The two substances, i.e. the mixed ester and the further plasticizer, may be present in different amounts in the plasticizer composition according to the invention. It should be clear that the two substances must be present in an amount in which they display an effect, i.e. where an altered plasticizing effect occurs. In a preferred embodiment of the present invention, the inventive mixture of triesters of the cyclohexane-1,2,4-tripropionic acid of the formula (1) below and the further plasticizer in the plasticizer composition are in a weight ratio (mixture of triesters:further plasticizer) of 1:99 to 99:1, preferably 10:90 to 90:10, more preferably 30:70 to 70:30.

The plasticizer composition according to the invention may further comprise at least one further plasticizer. The further plasticizer is preferably selected from the group consisting of adipates, benzoates, for example monobenzoates or glycol dibenzoates, chlorinated hydrocarbons (called chloroparaffins), citrates, epoxidized fatty acid esters, epoxidized vegetable oils, epoxidized acylated glycerides, furandicarboxylates, phosphates, succinates, sulfonamides, sulfonates, terephthalates, isophthalates, cyclohexanedicarboxylates, trimellitates, phthalates, triesters of 1,2,4-trialkylphosphoric acid excluding the compounds already present, and oligomeric or polymeric esters based on adipic acid, succinic acid or sebacic acid.

In a further-preferred embodiment, the at least one further plasticizer is selected from the group consisting of alkyl benzoates, alkylsulfonic esters of phenol, dialkyl adipates, glycerol esters, C4-C6 alkanoic esters of polyols, acetylated or non-acetylated trialkyl citrates, glycol dibenzoates, trialkyl esters of trimellitic acid, dialkyl terephthalates, dialkyl phthalates, dialkyl isophthalates, esters of furandicarboxylic acid, dialkanoyl esters of dianhydrohexitols (for example isosorbide), epoxidized fatty acid alkyl esters, polymer plasticizers, for example polyadipates, dialkyl esters of cyclohexane-1,2-, -1,3- or -1,4-dicarboxylic acid.

In a particularly preferred embodiment, the at least one further plasticizer present in the plasticizer composition according to the invention is selected from the group consisting of C8- to C13-alkyl benzoates, C4- to C10-dialkyl adipates, pentaerythritol tetravalerate, acetylated or non-acetylated trialkyl citrates having C4 to C9-alkyl groups, C4- to C10-trialkyl trimellitates, C4- to C9-dialkyl terephthalates, C4- to C13-dialkyl phthalates, especially C9- to C13-dialkyl phthalates and C4- to C10-dialkyl esters of cyclohexane-1,2-, -1,3- or -1,4-dicarboxylic acid.

The present invention accordingly provides a plasticizer composition comprising tributyl citrate and a mixture of triesters of cyclohexane-1,2,4-tripropionic acid of the following formula (1):

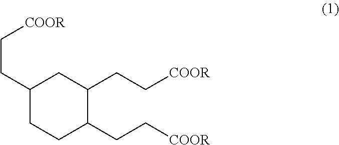

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and tributyl citrate in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising tripentyl citrate and a mixture of triesters of cyclohexane-1,2,4-tripropionic acid of the following formula (1):

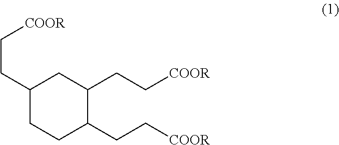

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and tripentyl citrate in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising acetyl tributyl citrate (ATBC) and a mixture of triesters of cyclohexane-1,2,4-tripropionic acid of the following formula (1):

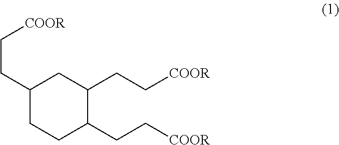

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and acetyl tributyl citrate (ATBC) in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising acetyl tripentyl citrate and a mixture of triesters of cyclohexane-1,2,4-tripropionic acid of the following formula (1):

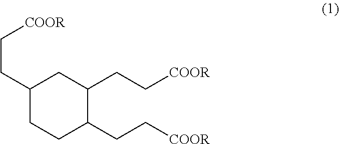

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and acetyl tripentyl citrate in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising diethylhexyl adipate (DEHA) and a mixture of triesters of cyclohexane-1,2,4-tripropionic acid of the following formula (1):

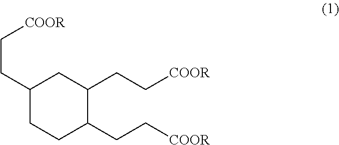

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and diethylhexyl adipate (DEHA) in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising diisononyl adipate (DINA) and a mixture of triesters of cyclohexane-1,2,4-tripropionic acid of the following formula (1):

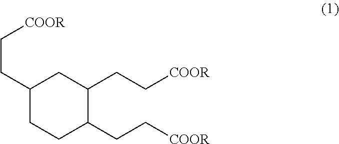

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and diisononyl adipate (DINA) in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising dibutyl terephthalate (DBT) and a mixture of triesters of cyclohexane-1,2,4-tripropionic acid of the following formula (1):

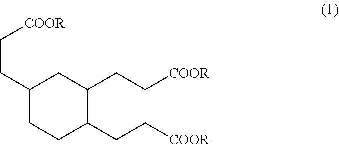

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and dibutyl terephthalate (DBT) in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising dipentyl terephthalate (DPT) and a mixture of triesters of cyclohexane-1,2,4-tripropionic acid of the following formula (1):

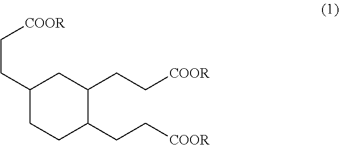

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and dipentyl terephthalate (DPT) in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising di-2-ethylhexyl terephthalate (DOTP) and a mixture of triesters of cyclohexane-1,2,4-tripropionic acid of the following formula (1):

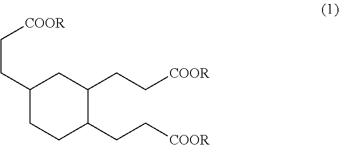

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and di-2-ethylhexyl terephthalate (DOTP) in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising diisononyl terephthalate (DINT) and a mixture of triesters of cyclohexane-1,2,4-tripropionic acid of the following formula (1):

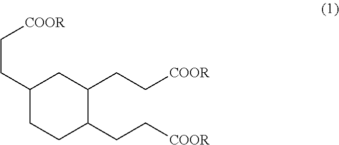

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and diisononyl terephthalate (DINT) in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising diisononyl cyclohexane-1,2-dicarboxylate (DINCH) and a mixture of triesters of cyclohexane-1,2,4-tripropionic acid of the following formula (1):

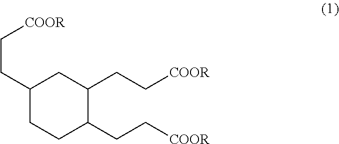

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and DINCH in the plasticizer composition are in a weight ratio (mixture of triesters:further plasticizer) of 1:99 to 99:1, preferably 10:90 to 90:10, more preferably 30:70 to 70:30 or 50:50. The present invention accordingly provides a plasticizer composition comprising diethylhexyl cyclohexane-1,4-dicarboxylate (1,4-DEHCH) and a mixture of triesters of cyclohexane-1,2,4-tripropionic acid of the following formula (1):

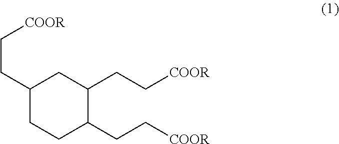

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and 1,4-DEHCH in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising diisononyl cyclohexane-1,4-dicarboxylate (DINCD) and a mixture of triesters of cyclohexane-1,2,4-tripropionic acid of the following formula (1):

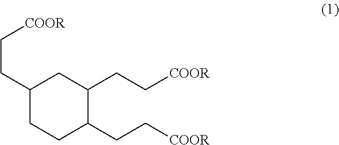

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and DINCD in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising di-2-ethylhexyl cyclohexane-1,2-dicarboxylate (1,2-DEHCH) and a mixture of triesters of cyclohexane-1,2,4-tripropionic acid of the following formula (1):

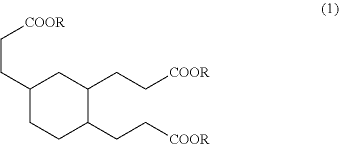

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and 1,2-DEHCH in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising triisononyl trimellitate (TINTM) and a mixture of triesters of cyclohexane-1,2,4-tripropionic acid of the following formula (1):

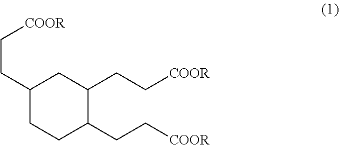

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and TINTM in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising tripentyl trimellitate (TPTM) and a mixture of triesters of cyclohexane-1,2,4-tripropionic acid of the following formula (1):

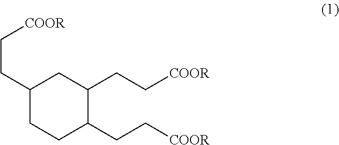

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and TPTM in the plasticizer composition are in a weight ratio (mixture of triesters:further plasticizer) of 1:99 to 99:1, preferably 10:90 to 90:10, more preferably 30:70 to 70:30 or 50:50.

The present invention accordingly provides a plasticizer composition comprising triethylhexyl trimellitate (TEHTM or TOTM) and a mixture of triesters of cyclohexane-1,2,4-tripropionic acid of the following formula (1):

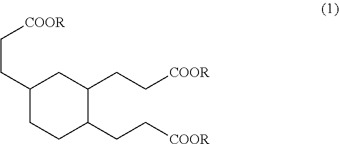

wherein 5 to 75 mol %, preferably 15 to 70 mol %, more preferably 20 to 65 mol %, of the R radicals in the mixture are methyl radicals and 25 to 95 mol %, preferably 30 to 85 mol %, more preferably 35 to 80 mol %, of the R radicals are a C2- to C10-alkyl radical, preferably a C5- to C9-alkyl radical, more preferably a C4- to C9-alkyl radical, most preferably a pentyl group or an isononyl group. The inventive mixture of triesters of the cyclohexane-1,2,4-tripropionic acid and TOTM in the plasticizer composition are in a weight ratio (mixture of triesters:further plasticizer) of 1:99 to 99:1, preferably 10:90 to 90:10, more preferably 30:70 to 70:30 or 50:50.

The plasticizer composition according to the invention may additionally contain an epoxidized oil or an epoxidized alkyl ester of a fatty acid. Epoxidized oils or corresponding esters can improve thermal stability and mechanical properties.

The epoxidized oil may be selected from the group consisting of epoxidized soybean oil, epoxidized castor oil, epoxidized linseed oil, epoxidized palm oil, epoxidized tall oil and mixtures thereof. Preference is given to using epoxidized soybean oil or epoxidized linseed oil in the plasticizer composition according to the invention. Particular preference is given to epoxidized soybean oil, also known by the acronym ESBO.

The epoxidized fatty acid esters can be prepared by transesterification of the abovementioned epoxidized oils with alcohols in the range of 1 to 10 carbon atoms, preferably 4 to 9 carbon atoms. Alternatively, the natural oil can first be transesterified and the double bonds of the fatty acid can then be epoxidized.

The epoxidized oil or the corresponding epoxidized fatty acid alkyl esters can be used in an amount of 1 to 150 parts by weight based on 100 parts by weight of the mixture of triesters of cyclohexane-1,2,4-tripropionic acid or the sum total of the mixture of triesters of cyclohexane-1,2,4-tripropionic acid and the further plasticizer. The epoxidized oil or the epoxidized fatty acid esters may also be present in the plasticizer composition in amounts of 2 to 125 parts by weight, 5 to 100 parts by weight, 10 to 80 parts by weight or 20 to 70 parts by weight, based in each case on 100 parts by weight of the mixture of triesters of cyclohexane-1,2,4-tripropionic acid or the sum total of the mixture of triesters of cyclohexane-1,2,4-tripropionic acid and the further plasticizer.

The present invention further provides a plastics composition comprising a plastic and a mixture of triesters of cyclohexane-1,2,4-tripropionic acid or the plasticizer composition comprising the mixture of triesters of cyclohexane-1,2,4-tripropionic acid and at least one further plasticizer. The plastics composition may additionally comprise the epoxidized oil and/or an epoxidized fatty acid alkyl ester and/or at least one additional plasticizer from the above list.

Suitable plastics are polymeric substances that are preferably selected from the group consisting of polyvinylchloride (PVC), homo- or copolymers based on ethylene, propylene, butadiene, vinyl acetate, glycidyl acrylate, glycidyl methacrylate, ethyl acrylate, butyl acrylate or methacrylate with alkoxy radicals from branched or unbranched alcohols having one to ten carbon atom(s), acrylonitrile or cyclic olefins, polyvinylidene chloride (PVDC), polyacrylates, in particular polymethylmethacrylate (PMMA), polyalkylmethacrylate (PAMA), polyureas, silylated polymers, fluoropolymers, especially polyvinylidene fluoride (PVDF), polytetrafluoroethylene (PTFE), polyvinylacetate (PVAc), polyvinylalcohol (PVA), polyvinylacetals, especially polyvinylbutyral (PVB), polystyrene polymers, especially polystyrene (PS), expandable polystyrene (EPS), acrylonitrile-styrene-acrylate (ASA), styrene-acrylonitrile (SAN), acrylonitrile-butadiene-styrene (ABS), styrene-maleic anhydride copolymer (SMA), styrene-methacrylic acid copolymer, polyolefins, especially polyethylene (PE) or polypropylene (PP), thermoplastic polyolefins (TPO), polyethylene-vinyl acetate (EVA), polycarbonates, polyethylene terephthalate (PET), polybutylene terephthalate (PBT), polyoxymethylene (POM), polyamide (PA), polyethylene glycol (PEG), polyurethane (PU), thermoplastic polyurethane (TPU), polysulfides (PSu), biopolymers, especially polylactic acid (PLA), polyhydroxybutyral (PHB), polyhydroxyvaleric acid (PHV), polyesters, starch, cellulose and cellulose derivatives, especially nitrocellulose (NC), ethylcellulose (EC), cellulose acetate (CA), cellulose acetate/butyrate (CAB), rubber and silicones.

In a preferred embodiment, the plastic in the plasticizer composition is selected from the group consisting of polyvinylchloride (PVC), polyalkylmethacrylate (PAMA), polyvinylbutyral (PVB), polyurethane, polysulfides, polylactic acid (PLA), polyhydroxybutyral (PHB), nitrocellulose and copolymers of vinyl chloride with vinyl acetate or with butyl acrylate. Among these, particular preference is given to PVC as plastic in the plastics composition according to the invention.

The amount of the plasticizer composition according to the invention in the plastics composition is preferably 5 to 150 parts by weight, preferably 10 to 120 parts by weight, more preferably 15 to 110 parts by weight and most preferably 20 to 100 parts by weight per 100 parts by weight of the plastic.

The plastics composition may contain additional additives as well as the ingredients mentioned. Examples of additional additives are rheological additives with which the viscosity of the plastics composition can be reduced. Examples of known rheological additives are the products available under the trade names VISCOBYK®-5120, VISCOBYK®-5130 and VISCOBYK®-4041. The additives may be present in the plastics composition in a proportion of 1 to 12, preferably 2 to 10, parts by weight per 100 parts by weight of PVC.

In addition, the plastics composition may comprise one or more thermal stabilizer(s). Suitable thermal stabilizers are lead salts, organotin compounds, barium/zinc compounds, cadmium compounds or zinc compounds, calcium/zinc stabilizers and organic-based stabilizers (“OBS”). Preferred thermal stabilizers are barium/zinc compounds, calcium/zinc stabilizers and organic-based stabilizers (“OBS”). The proportion of the stabilizer(s) in the plastics composition is preferably 1 to 6 parts by weight per 100 parts by weight of PVC.

Further additives present in the plastics composition may additionally also be fillers, pigments, blowing agents and lubricants.

The plastics composition according to the invention is preferably part of an adhesive, of a sealing compound, of a coating compound, of a varnish, of a paint, of a plastisol, of a dryblend, of a foam, of an imitation leather, of a floor covering, especially the top layer or foam layer thereof, of a roofing membrane, of an underbody protection, of a fabric coating, of a cable, of a wire insulation, of a hose, of an extruded article, of a film, of an article in the field of automotive interiors, of a wallpaper, of an ink, of a toy, of a contact sheet, of a food packaging or of a medical article, especially of a tube or of a blood bag.

The present invention therefore also provides for the use of the plastics composition in adhesives, sealing compounds, coating compounds, varnishes, paints, plastisols, foams, imitation leathers, floor coverings, especially the top layer and foam layer, roofing membranes, underbody protection, fabric coatings, cables, wire insulation, hoses, extruded articles, films, in the automotive interior sector, in wallpaper, inks, toys, contact sheets, food packaging or medical articles, especially in tubes or blood bags.

The present invention is illustrated hereinafter by examples. The examples which follow are intended to illustrate the invention without restricting its scope of application as apparent from the description and the embodiments.

### EXAMPLES

### Example 1—Preparation of trimethyl cyclohexane-1,2,4-tripropionate (Me-Tc)

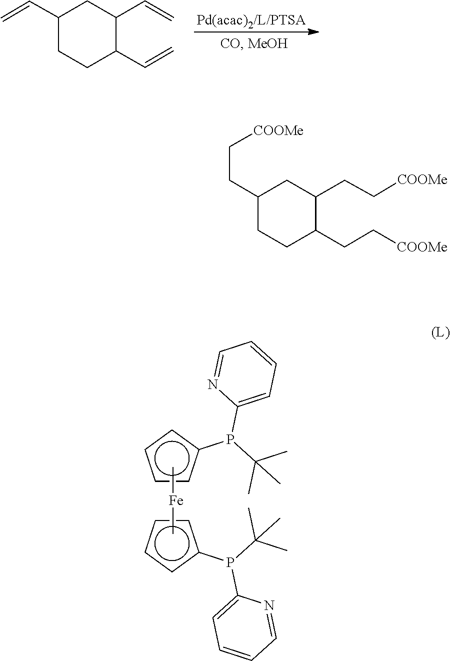

A 100 ml stainless steel autoclave was charged with [Pd(acac)2](15.2 mg, 0.1 mol %), the catalyst L (103 mg, 0.4 mol %) and para-toluenesulfonic acid (PTSA, 143 mg, 1.5 mol %) under an argon atmosphere. Then MeOH (30 ml) and trivinylcyclohexane (8.1 g, 50 mmol) were injected by syringe. The autoclave was flushed three times with CO and then CO was injected to a pressure of 40 bar. The reaction was carried out at 110° C. for 10 h. Then, the autoclave was cooled to room temperature and depressurized. The desired product was purified by distillation (165° C. at 10-3 bar) and characterized by ¹H NMR, ¹³C NMR and HR-MS (15.6 g, 91% yield).

### Examples 2a and 2b—Preparation of mixed esters of cyclohexane-1,2,4-tricarboxylic Acid with Methanol and Pentanol

The trimethyl ester (Me-Tc) prepared according to Example 1 was transesterified in a typical transesterification apparatus in the laboratory with n-pentanol to give the MPe-Tc product. The transesterification apparatus consists of a glass flask equipped with a stirrer, temperature sensor, distillation column, vacuum divider and distillation setup.

For example 2a, the transesterification apparatus was first charged with 150 g of n-pentanol and 484 g of M-Tc. Thereafter, the whole apparatus was purged with nitrogen, and then 0.89 g of tetra(n-butyl)titanium (TNBT) as catalyst was added. The reaction was then started by heating. The reaction temperature was 130-240° C., i.e. it rose during the reaction. Methanol formed in the course of transesterification was removed from the system via the column and distillation apparatus (top temperature 65° C.). The C5 alcohol which was separated from the methanol in the column was returned to the reaction mixture as reflux. When no more occurrence of distillate was observed, the reaction was ended.

After the reaction, the excess alcohol was distilled off at 160° C. under reduced pressure. The acid number of the reaction mixture was then determined, and the reaction mixture was then neutralized by adding 3 times the stoichiometric amount of 10% NaOH. Next the mixture was stripped at 160° C. with nitrogen for 2 h. In the last step, the product is filtered through a 0.5 μm PTFE filter and Perlite as filter aid to give a clear filtrate.

The synthesis for example 2 b was performed analogously with 222 g of n-pentanol, 378 g of M-Tc and 0.68 g of TNBT.

The exact proportion of methyl and pentyl groups in mol % was determined by 1H NMR after the reactions, and the proportions of the different esters by GC in area %

TABLE 1
Reactant ratios and compositions of the triesters

| | Equivalents of n- | Proportion of | Proportion of |
|---|---|---|---|
| | pentanol per mole of | methyl ester in | pentyl ester in |
| | Me—Tc in the synthesis | the product | the product |
| 2a* | 1.2 | 59 | 41 |
| 2b* | 2.3 | 25 | 75 |

| | area % of | area % of dimethyl | area % of | area % of |
|---|---|---|---|---|
| | trimethyl | monopentyl | monomethyl | tripentyl |
| | ester | ester | dipentyl ester | ester |
| 2a* | 15.6 | 41.2 | 34.2 | 8.8 |
| 2b* | 1.1 | 11.6 | 40.8 | 46.4 |

*according to the invention

### Examples 3a and 3b—Preparation of Mixed Esters of cyclohexane-1,2,4-tricarboxylic Acid with Methanol and Isononanol

The trimethyl ester (Me-Tc) prepared according to Example 1 was transesterified with isononanol to give the In—Tc product in a typical laboratory transesterification apparatus as in Example 2a/2b.

For example 3a, the transesterification apparatus was first charged with 190 g of isononanol (INA, manufacturer: Evonik Oxeno GmbH & Co. KG) and 376 g of M-Tc. Subsequently, the whole apparatus was purged with nitrogen. Next, 0.37 g of tetra(n-butyl)titanium (TNBT) was added as catalyst. The reaction was then started by heating. The reaction temperature was 130-240° C., i.e. it rose during the reaction. Methanol formed in the course of transesterification was removed from the system via the column and distillation apparatus (top temperature 65° C.). INA which was separated from the methanol in the column was returned to the reaction mixture as reflux. A slight vacuum was applied occasionally in order to obtain sufficient distillate. When no more occurrence of distillate was observed, the reaction was ended.

After the reaction, the excess alcohol was distilled off at 180° C. under reduced pressure. The acid number of the reaction mixture was then determined, and the reaction mixture was then neutralized by adding 3 times the stoichiometric amount of 10% NaOH. Next the mixture was stripped at 180° C. with nitrogen for 2 h. In the last step, the product is filtered through a 0.5 μm PTFE filter and Perlite as filter aid to give a clear filtrate.

The synthesis for example 3b was performed analogously with 288 g of isononanol, 301 g of M-Tc and 0.45 g of TNBT.

The exact proportion of methyl and isononyl groups in mol % was determined by 1H NMR after the reactions, and the proportions of the different esters by GC in area %

TABLE 2
Reactant ratios and compositions of the triesters

| | Equivalents of n- | Proportion of | Proportion of |
|---|---|---|---|
| | isononanol per mole of | methyl ester | pentyl ester in |
| | Me—Tc in the synthesis | in the product | the product |
| 3a* | 1.2 | 60 | 40 |
| 3b* | 2.3 | 23 | 77 |

| | area % of | area % of dimethyl | area % of | area % of |
|---|---|---|---|---|
| | trimethyl | monoisononyl | monomethyl | triisononyl |
| | ester | ester | diisononyl ester | ester |
| 3a* | 12.8 | 42.1 | 36.3 | 8.2 |
| 3b* | 0.5 | 9.4 | 42.4 | 46.8 |

*according to the invention

### Example 4—Production of Plastisols

PVC plastisols were produced, as used, for example, for manufacture of topcoat films for floor coverings. The figures in the plastisol recipe are each in parts by weight (phr). The recipe of the polymer composition is listed in Tables 3 and 4.

TABLE 3
Overview of the plastisols produced (Part 1)

| | 1 | 2* | 3* |
|---|---|---|---|
| Plastisol | phr | phr | phr |
| PVC (Vestolit ® P 1430 K 70 Ultra; from | 100 | 100 | 100 |
| Vestolit) | | | |
| Me—Tc | 50 | | |
| C1/C5 mixed ester 59/41 (Ex. 2a) | | 50 | |
| C1/C5 mixed ester 25/75 (Ex. 2b) | | | 50 |
| Epoxidized soybean oil as co-stabilizer | 3 | 3 | 3 |
| (Edenol ® D81, from Emery) | | | |
| Thermal stabilizer based on Ca/Zn | 2 | 2 | 2 |
| (Reagent MBL 197/9 PF) | | | |

*= composition according to the invention
phr = parts per hundred parts resin

TABLE 4
Overview of the plastisols produced (Part 2)

| | 4* | 5* |
|---|---|---|
| Plastisol | phr | phr |
| PVC (Vestolit ® P 1430 K 70 Ultra; from | 100 | 100 |
| Vestolit) | | |
| C1/C9 mixed ester 60:40 (Ex. 3a) | 50 | |
| C1/C9 mixed ester 23:77 (Ex. 3b) | | 50 |
| Epoxidized soybean oil as co-stabilizer | 3 | 3 |
| (Edenol ® D81, from Emery) | | |
| Thermal stabilizer based on Ca/Zn | 2 | 2 |
| (Reagent MBL 197/9 PF) | | |

*= composition according to the invention
phr = parts per hundred parts resin

First the liquid constituents and then the pulverulent constituents were weighed out into a PE cup. The mixture was stirred manually with a spatula in such a way that there was no longer any unwetted powder. The closed cup was placed in the mount in the Speedmixer and mixed and deaerated. After the mixing was finished, the temperature of the plastisol was measured using an IR thermometer. Then the plastisol was immediately equilibrated to 25.0° C. in a climate-controlled cabinet for further studies.

### Example 5—Measurement of the Viscosities of Plastisols

The viscosity of the plastisols produced in Example 4 was measured with a Physica MCR 101 rheometer (from Anton Paar Germany GmbH) with the aid of the associated software, using the rotation mode and the CC27 measuring system.

The measurement involved the following points:
preliminary shear at 100 s⁻¹ for a period of 60 seconds, during which no measurements were taken
downward shear rate ramp from 200 s⁻¹ to 0.1 s⁻¹. 30 measurement points were recorded, each with a measurement point duration of 10 seconds.

The measurements were conducted at room temperature. The results of the viscosity measurements for the plastisols produced in Example 4 with the plasticizers or plasticizer mixtures specified in each case are shown in Table 5 (measurement for 2 h and 24 h at a shear rate of 100 s⁻¹).

TABLE 5
Results of viscosity measurement

| | Viscosity at 100 s⁻¹ | Viscosity at 100 s⁻¹ | Rise from 2 h |
|---|---|---|---|
| Recipe | after 2 h/Pa*s | after 24 h/Pa*s | to 24 h/% |
| 1 | 14.9 | 19.5 | 31.4 |
| 2* | 7.1 | 9.2 | 28.5 |
| 3* | 5.2 | 6.2 | 18.2 |
| 4* | 10.7 | 12.0 | 12.7 |
| 5* | 13.5 | 12.3 | −8.8 |

*= inventive

Compared to the trimethyl ester, the inventive mixed esters 2* to 5* have both a lower viscosity level and a smaller change in viscosity (percentage change from 2 h to 24 h), which enables better processibility of the plastisol.

### Example 6—Production of Films

The plastisols produced in Example 4 were each processed to give films of thickness 1 mm. For this purpose, first of all, high-gloss release paper (from Sappi, Italy) was trimmed to a size of 30×44 cm and inserted in the clamping frame of the LTSV coating installation for the Mathis oven. The clamping frame was subsequently placed onto the guide frame, the Mathis oven (model LTF) was adjusted to 200° C. and, when this temperature was attained, the frame was preheated for 15 seconds. The coating bar was subsequently inserted into the clamping means and the coating bar gap was adjusted via preliminary experiments such that the film thickness on conclusion of gelation was 1 mm (+/−0.05 mm). An adhesive strip was mounted on the leading edge of the paper in order to catch excess plastisol. The plastisol was then applied in front of the coating knife, and spread by drawing of the guide frame with the coating knife over the clamped released paper (at a speed of 3 m/min). The coating knife was then removed and the adhesive strip with the excess plastisol was removed. The clamping frame was then moved into the oven. After gelation (2 minutes at 200° C.), the frame was moved back out of the oven and, after cooling, the film was removed from the paper.

### Example 7—Determination of Loss of Mass (Activated Carbon Method)

In accordance with DIN EN ISO 176 (Method B), three circular discs with a diameter of 5 cm were in each case punched out of the PVC films produced and first conditioned in a desiccator at room temperature for 16 hours. The sample circles were weighed out on an analytical balance and placed in a wire basket closed with a bulldog clip. The wire baskets were then placed into an activated carbon-filled tin can such that there was about 130 ml of activated carbon in each case between the individual baskets or the basket and the lid (perforated) or base.

The filled tin cans were positioned in a preheated heating cabinet (120° C.) in such a way that the ventilator in the cabinet was not blocked and the cans were not in contact with one another. Air exchange and ventilation flap were each set at 10%. After 72 hours, the samples were taken out of the cabinet again and cooled down, then the individual samples were taken out of the baskets, conditioned for another 16 hours in a desiccator and then reweighed on an analytical balance. The resultant difference in mass results from the loss of plasticizer. The average was formed from the three differences in mass in each case, and the percentage loss of plasticizer was calculated. The volatility results are shown in Table 6.

TABLE 6
Loss of mass of the test specimens

| Film number | Loss of mass after 3 days in % |
|---|---|
| 1 | 7.3 |
| 2* | 3.0 |
| 3* | 1.7 |
| 4* | 2.5 |
| 5* | 1.5 |

*= inventive

The loss of mass of films comprising the plasticizers according to the invention is lower than that of films comprising the trimethyl esters.

### Example 8—Measurement of Glass Transition Temperature

The glass transition temperature was determined by DMTA measurements in accordance with DIN 65583 using a type MCR 302 rheometer from Anton Paar. Under constant dynamic mechanical conditions (1 Hz, deformation 0.3%), the viscoelastic properties of the films were recorded as a function of temperature (temperature ramp from −100 to +50° C.) and the storage modulus, the loss modulus and the loss factor were determined. The maximum of the loss module is evaluated here as the glass transition temperature TG (see Table 7).

TABLE 7
Glass transition temperatures of the films

| Film number | Tg in ° C. |
|---|---|
| 1 | −18.6 |
| 2* | −25.2 |
| 3* | −30.5 |
| 4* | −27.3 |
| 5* | −37.4 |

*= inventive

The higher the C5 or C9 content of the films according to the invention, the lower the glass transition temperatures.

1. A mixture of triesters of cyclohexane-1,2,4-tripropionic acid of the following formula (1): 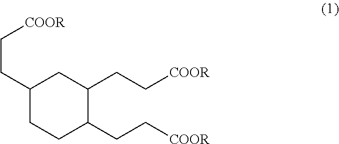
wherein 5 to 75 mol % of the R radicals in the mixture are methyl radicals and 25 to 95 mol % of the R radicals are a C2- to C10-alkyl radical.

2. The mixture according to claim 1, wherein 15 to 70 mol % of the R radicals in the mixture are methyl radicals and 30 to 85 mol % of the R radicals are a C2- to C10-alkyl radical.

3. The mixture according to claim 1, wherein the C2- to C10-alkyl radical is a C5- to C9-alkyl radical.

4. A plasticizer composition, comprising
a mixture of triesters of cyclohexane-1,2,4-tripropionic acid of the following formula (1): 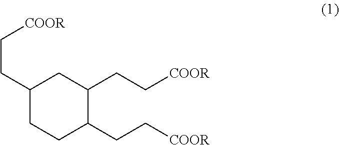
wherein
5 to 75 mol % of the R radicals in the mixture are methyl radicals and 25 to 95 mol % of the R radicals are a C2- to C10-alkyl radical, and
at least one further plasticizer.

5. The plasticizer composition according to claim 4, wherein the mixture of triesters of the cyclohexane-1,2,4-tripropionic acid of the formula (1) and the further plasticizer in the plasticizer composition are in a weight ratio (mixture of triesters of the cyclohexane-1,2,4-tripropionic acid of the formula (1): further plasticizer) of 1:99 to 99:1.

6. The plasticizer composition according to claim 4, wherein the at least one further plasticizer is selected from the group consisting of adipates, benzoates, chlorinated hydrocarbons, citrates, epoxidized fatty acid esters, epoxidized vegetable oils, epoxidized acylated glycerides, furandicarboxylates, phosphates, succinates, sulfonamides, sulfonates, terephthalates, isophthalates, cyclohexanedicarboxylates, trimellitates, phthalates, and oligomeric or polymeric esters based on adipic acid, succinic acid or sebacic acid.

7. The plasticizer composition according to claim 4, wherein the plasticizer composition additionally comprises an epoxidized oil or an epoxidized alkyl ester of a fatty acid.

8. The plasticizer composition according to claim 7, wherein the epoxidized oil is selected from the group consisting of epoxidized soybean oil, epoxidized castor oil, epoxidized linseed oil, epoxidized palm oil, epoxidized stearate, epoxidized oleate, epoxidized tall oil, epoxidized linoleate and mixtures thereof.

9. The plasticizer composition according to claim 8, wherein the epoxidized oil is epoxidized soybean oil or epoxidized linseed oil.

10. The plasticizer composition according to claim 7, wherein the epoxidized oil is present in the plasticizer composition in an amount of 1 to 150 parts by weight based on 100 parts by weight of a sum total of a compound of formula (1) and a dialkyl terephthalate.

11. A plastics composition, comprising:
the mixture according to claim 1, and
a plastic.

12. The plastics composition according to claim 11, wherein a proportion of the mixture is 5 to 150 parts by weight per 100 parts by weight of plastic.

13. The plastics composition according to claim 11, wherein the plastic is selected from the group consisting of polyvinylchloride (PVC), polyalkylmethacrylate (PAMA), polyvinylbutyral (PVB), polyurethane, polysulfides, polylactic acid (PLA), polyhydroxybutyral (PHB), nitrocellulose and copolymers of vinyl chloride with vinyl acetate or with butyl acrylate.

14. The plastics composition according to claim 13, wherein the plastic is polyvinylchloride (PVC).

15. A product, comprising:
the plastics composition according to claim 11,
wherein the product is selected from the group consisting of adhesives, sealing compounds, coating compounds, varnishes, paints, plastisols, foams, imitation leathers, floor coverings, roofing membranes, underbody protection, fabric coatings, cables, wire insulation, hoses, extruded articles, films, automotive interior components, wallpaper, inks, toys, contact sheets, food packaging, and medical articles.
